SEQUENCE LISTING (6 sequences)

SEQ ID NO: 1 - DNA, 945 bp, Artificial Sequence
  nucleotide sequence encoding cloning vector pBS 35S attR-hRluc, complete sequence
atggcttcgaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccgctagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggccagacatcgaggaggacatcgccctgatcaagagcgaggagggcgagaagatggtgctggagaacaacttcttcgtggagaccatgctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagatgttcatcgagagcgaccccggcttcttcagcaacgccatcgtggagggcgccaagaagttccccaacaccgagttcgtgaaggtgaagggcctgcacttcagccaggaggacgcccccgacgagatgggcaagtacatcaagagcttcgtggagagagtgctgaagaacgagcagagatctatctag

SEQ ID NO: 2 - DNA, 894 bp, Bacillus megaterium
atgagtaacaagtatagagttagaaaaaacgtattacatcttaccgacacggaaaaaagagattttgttcgtaccgtgctaatactaaaggaaaaagggatatatgaccgctatatagcctggcatggtgcagcaggtaaatttcatactcctccgggcagcgatcgaaatgcagcacatatgagttctgcttttttaccgtggcatcgtgaataccttttacgattcgaacgtgaccttcagtcaatcaatccagaagtaacccttccttattgggaatgggaaacggacgcacagatgcaggatccctcacaatcacaaatttggagtgcagattttatgggaggaaacggaaatcccataaaagattttatcgtcgataccgggccatttgcagctgggcgctggacgacgatcgatgaacaaggaaatccttccggagggctaaaacgtaattttggagcaacgaaagaggcacctacactccctactcgagatgatgtcctcaatgctttaaaaataactcagtatgatacgccgccttgggatatgaccagccaaaacagctttcgtaatcagcttgaaggatttattaacgggccacagcttcacaatcgcgtacaccgttgggttggcggacagatgggcgttgtgcctactgctccgaatgatcctgtcttctttttacaccacgcaaatgtggatcgtatttgggctgtatggcaaattattcatcgtaatcaaaactatcagccgatgaaaaacgggccatttggtcaaaactttagagatccgatgtacccttggaatacaacccctgaagacgttatgaaccatcgaaagcttgggtacgtatacgatatagaattaagaaaatcaaaacgttcctcataa

SEQ ID NO: 3 - DNA, 723 bp, Artificial Sequence
  nucleotide sequence encoding Super folder GFP
catatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaaaggtattgattttaaagaagatggaaacattctcggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggatcc

SEQ ID NO: 4 - DNA, 2910 bp, Artificial Sequence
  nucleotide sequence encoding  p50 (NF-kB subunit)
atggcagaagatgatccatatttgggaaggcctgaacaaatgtttcatttggatccttctttgactcatacaatatttaatccagaagtatttcaaccacagatggcactgccaacagatggcccataccttcaaatcttagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtagtgaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaagcagccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagtagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacgatcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacacgggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 5 - DNA, 2910 bp, Artificial Sequence; identical to SEQ ID NO: 4
  nucleotide sequence of Pet3 Plasmid encoding for Pseudomonas exotoxin A (PE)

SEQ ID NO: 6 - DNA, 1900 bp, Artificial Sequence
  nucleotide sequence encoding Pseudomonas exotoxin A with histidine tag
ctagaaataaagaaggagatataccatgggcagcagccatcatcatcatcatcacagcagcgccgaagaagctttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatcccggaccaagaacaggcgatcagcgccctgccggactacgccagccagcccgggcaaccgccgcgcgaggacctgtag

(19) United States
(12) Patent Application Publication
(10) Pub. No.: US 2017/0198326 A1
(43) Pub. Date: Jul. 13, 2017

(54) METHODS AND KITS FOR CELL-FREE TRANSCRIPTION AND TRANSLATION

(71) Applicant: Technion Research & Development Foundation Limited, Haifa (IL)

(72) Inventors: Avraham SCHROEDER, Binyamina (IL); Janna SHAINSKY-ROITMAN, Yokneam Ilit (IL); Mor GOLDFEDER, Ramat Yishai (IL); Nitzan KRINSKY, Kiryat Ata (IL); Yuval SHOHAM, Haifa (IL)

(21) Appl. No.: 15/324,756

(22) PCT Filed: Jul. 8, 2015
(86) PCT No.: PCT/IL2015/050710

§ 371 Date: Jan. 9, 2017

Related U.S. Application Data
(60) Provisional application No. 62/021,748, filed on Jul. 8, 2014.

(51) Int. Cl. C12P 21/00; C12N 1/06; C12N 9/12; C12P 19/34

(52) U.S. Cl. CPC C12P 21/00; C12P 19/34; C12N 1/066; C12N 9/1247; C12Y 207/07006

(57) ABSTRACT

A method for cell-free protein synthesis, the method comprising synthesizing RNA or a protein of interest in a reaction mixture comprising: a template DNA encoding the RNA or protein of interest; and a biological extract of a protease-deficient bacterial cell that has been genetically modified to express a heterologous RNA polymerase, the extract further comprising components necessary for transcription and translation of the protein. Also provided are a method for producing a reaction mixture for cell-free protein synthesis and kits for executing these methods.

### FIELD AND BACKGROUND OF THE INVENTION

The present invention, in some embodiments thereof, relates to methods and kits for cell-free transcription and translation.

Proteins have great industrial and therapeutic importance. Bacteria, remain the most efficient systems for transcribing and translating genetic code into functional proteins. A more advance platform is based on nanoparticles that contain cell-free extracts and can be remotely triggered to synthesize therapeutic proteins within diseased organs.¹

There is a growing need for cell-free protein production processes for research and therapeutic applications.² Applications include the study of protein-protein interactions, developing industrial enzymes, or investigating new drug mechanisms.³ In addition, due to the fact that many therapeutic proteins are toxic or can't be properly folded within cells they require cell-free system.⁴

As mentioned, to date, most proteins are produced using cell extracts that were manufactured from bacterial, plant or mammalian cells.⁵ The most commonly used cell extract is the ‘Escherichia coli S30’, developed initially by Nirenberg and Matthaei⁶ and then standardized by Zoubay⁷ and Pratt⁸. The acronym S30 stands for the 30,000 g centrifugation step that separates the transcription/translation machineries from unnecessary heavy cellular components.⁵ The S30 extract can be used in its crude form, supplemented with essential amino acids, transcription factors and salts,⁹ or, in a form of purified machinery that is reconstituted with all the necessary protein-production components in accurate molar ratios.^{9,10} These processes were licensed by Promega and New England Biolabs (NEB) Inc., respectively.

The existing processes are lengthy and require expensive tools (such as the 30 g centrifuge) and reagents.

Additional background art includes:
1 Schroeder, A. et al. Remotely-activated protein-producing nanoparticles. Nano Lett, doi:10.1021/n12036047 (2012).
2 Carlson, E. D., Gan, R., Hodgman, C. E. & Jewett, M. C. Cell-free protein synthesis: Applications come of age. Biotechnol. Adv. 30, 1185-1194, doi:http://dx(dot)doi(dot)org/10.1016/j.biotechadv..2011.09.016 (2012).
3 Whittaker, J. Cell-free protein synthesis: The state of the art. Biotechnol. Lett. 35, 143-152, doi:10.1007/s10529-012-1075-4 (2013).
4 Bernhard, F. & Tozawa, Y. Cell-free expression-making a mark. Curr. Opin. Struct. Biol. 23, 374-380, doi:http://dx(dot)doi(dot)org/10.1016/j.sbi.2013.03.012 (2013).
5 Spirin, A. S. & Swartz, J. R. in Cell-Free Protein Synthesis 1-34 (Wiley-VCH Verlag GmbH & Co. KGaA, 2008).
6 Nirenberg, M. W. in Methods Enzymol. Vol. Volume 6 17-23 (Academic Press, 1963).
7 Zubay, G. In Vitro Synthesis of Protein in Microbial Systems. Annu. Rev. Genet. 7, 267-287, doi:doi:10.1146/annurev.ge.07.120173.001411 (1973).
8 Nevin, D. E. & Pratt, J. M. A coupled in vitro transcription-translation system for the exclusive synthesis of polypeptides expressed from the T7 promoter. FEBS Lett. 291, 259-263, doi:http://dx(dot)doi(dot)org/10.1016/0014-5793(91)81297-L (1991).
9 Pratt, J. M. in Transcription and Translation a practical approach (eds B. D. Hames & S. J. Higgins) Ch. 7, 179-209 (IRL Press 1984).
10 Shimizu, Y. et al. Cell-free translation reconstituted with purified components. Nat. Biotechnol. 19, 751-755, doi:http://www(dot)nature(dot)com/nbt/journal/v19/n8/suppinfo/nbt0801_751_S1(dot)html (2001).
11 Sunami, T. et al. Femtoliter compartment in liposomes for in vitro selection of proteins. Analytical Biochemistry 357, 128-136 (2006).
12 LB (Luria-Bertani) liquid medium. Cold Spring Harbor Protocols 2006 (2006).
13 Terrific Broth. Cold Spring Harbor Protocols 2006, pdb.rec8620 (2006).
14. Ivanir 2009 “Development of an efficient cell-free translation system” Research Thesis.

### SUMMARY OF THE INVENTION

According to an aspect of some embodiments of the present invention there is provided a method for cell-free RNA or protein synthesis, the method comprising synthesizing an RNA or protein of interest in a reaction mixture comprising:

a template DNA encoding the RNA or protein of interest; and

a biological extract of a protease-deficient bacterial cell that has been genetically modified to express a heterologous RNA polymerase, the extract further comprising components necessary for transcription and translation of the protein.

According to an aspect of some embodiments of the present invention there is provided a method of producing a reaction mixture for cell-free protein synthesis, the method comprising:

(a) growing a culture of protease-deficient bacterial cells that have been genetically modified to express a heterologous RNA polymerase under conditions which allow expression of the RNA polymerase;

(b) disrupting the protease-deficient bacterial cells to obtain a cell preparation which comprises genomic DNA and broken cells; the disrupting being under conditions which retain functionality of components necessary for transcription and translation of the protein;

(c) removing the genomic DNA; and

(d) freezing the cell preparation following the removing.

According to some embodiments of the invention, the method further comprises encapsulating the cell preparation in lipid vesicles.

According to some embodiments of the invention, the RNA polymerase is expressed from a plasmid.

According to some embodiments of the invention, the disrupting is effected using a pressure homogenizer.

According to some embodiments of the invention, the method does not comprise dialyzing the cell preparation following the disrupting.

According to some embodiments of the invention, the method does not comprise freezing and thawing the bacterial cells following step (a) and prior to step (b).

According to some embodiments of the invention, the method does not comprise adding a protease inhibitor to the cell preparation.

According to some embodiments of the invention, the removing comprises centrifuging at lower than 30,000×g.

According to an aspect of some embodiments of the present invention there is provided a composition comprising an extract of protease-deficient bacterial cells that have been genetically modified to express a heterologous RNA polymerase, the extract comprising components necessary for transcription and translation and being devoid of genomic DNA.

According to an aspect of some embodiments of the present invention there is provided a kit for cell-free RNA or protein synthesis comprising an extract of protease-deficient bacterial cells that have been genetically modified to express a heterologous RNA polymerase, the extract comprising components necessary for transcription and translation.

According to some embodiments of the invention, the kit further comprises at least one of:

(i) lipid which are preformed as or capable of forming lipid vesicles;

(ii) a control DNA template;

(iii) amino acids;

(iv) rNTPs;

(v) H₂O;

(vi) salt; and

(vii) an ATP-regenerating system.

According to some embodiments of the invention, the extract comprises a plasmid which encodes the RNA polymerase.

According to some embodiments of the invention, the control DNA template is separately packaged from the extract.

According to some embodiments of the invention, each of the (i)-(vii) is separately packaged.

According to some embodiments of the invention, at least two of the (i)-(vii) are separately packaged.

According to some embodiments of the invention, the components necessary for transcription and translation are selected from the group consisting of tRNAs, ribosomes and transcription factors.

According to some embodiments of the invention, the biological extract is present in the reaction mixture at a concentration of 20-40% (v/v).

According to some embodiments of the invention, the biological extract is present in the reaction mixture at a concentration of 30% (v/v).

According to some embodiments of the invention, the reaction mixture further comprises amino acids, rNTPs, H₂O, salt and an ATP-regenerating system.

According to some embodiments of the invention, the reaction mixture comprises polyethylene glycol.

According to some embodiments of the invention, the template DNA is a circular DNA.

According to some embodiments of the invention, the template DNA comprises a promoter for the RNA polymerase being operably linked to a nucleic acid sequence encoding the RNA or protein.

According to some embodiments of the invention, the RNA polymerase is a DNA-dependent RNA polymerase.

According to some embodiments of the invention, the DNA-dependent RNA polymerase is a phage RNA polymerase.

According to some embodiments of the invention, the phage RNA polymerase is a T7 or SP6 RNA polymerase.

According to some embodiments of the invention, the T7 is encoded from pAR1219.

According to some embodiments of the invention, the salt is selected from the group consisting of potassium, magnesium and ammonium.

According to some embodiments of the invention, the reaction mixture comprises 10-20 mM magnesium salt, 40-60 mM potassium salt and 100-200 nM ammonium salt.

According to some embodiments of the invention, the reaction mixture is as listed in Table 2 above.

According to some embodiments of the invention, the synthesizing is effected in batch, continuous flow, or semi-continuous flow.

According to some embodiments of the invention, the protease-deficient bacterial cells are selected from the group consisting of BL21(DE3), BL21(DE3) CodonPlus RIL and variants thereof.

According to some embodiments of the invention, the protein is a membranal protein.

According to some embodiments of the invention, the RNA is a silencing agent.

According to some embodiments of the invention, the lipid vesicles comprise liposomes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

FIG. 1 is a scheme comparing the lysate preparation according to some embodiments of the invention with that of prior art taught by Pratt, J. M. in Transcription and Translation a practical approach (eds B. D. Hames & S. J. Higgins) Ch. 7, 179-209 (IRL Press 1984).

FIG. 2 is a scheme describing the process of manufacturing a potent bacterial lysate for cell free protein production according to some embodiments of the invention, further elaborated herein below in the Examples section which follows. It aims to describe a general embodiment of the invention.

FIG. 3 is a bar graph comparing the protein producing system of some embodiments of the invention to the commercially available system (Promega). Renilla luciferase was produced by both systems after 90 min of incubation, A two-fold increase in yield is evident in the present method, as compared to that of the prior art.

FIG. 4 is a bar graph showing the thermo-stability of the present system according to some embodiments of the invention in sfGFP synthesis in different temperatures.

FIG. 5 is a bar graph showing the activity of the extract in protein sfGFP synthesis when encapsulated according to some embodiments of the invention. Protein production is evident in various particle sizes.

FIG. 6 is a graph showing sfGFP production kinetics in cell free system according to some embodiments of the invention.

FIGS. 7A-C show the production of sfGFP in cell free and in particles. FIG. 7A—Production of GFP in the present cell free system; FIG. 7B—Production of GFP in DMPC:Cholesterol particles; FIG. 7C—sfGFP production in DMPC:Cholesterol particles (left panel—time zero; right panel—following 10 min).

FIG. 8 shows the production of tyrosinase from Bacillus megaterium using the present cell-free system according to some embodiments of the invention. The visualization was done using Tecan infinite pro200 spectrophotometer at absorbance of 475 nm.

FIG. 9 is a SDS-PAGE visualized by autoradiography. The image shows production of p50 (NF-Kβ subunit) in the cell-free system according to some embodiments of the invention.

FIG. 10A is a Western Blot analysis of cell free productions of Pseudomonas exotoxin (PE) using the present cell free system. Anti-PE antibody was used as the primary antibody. A cell-reaction without DNA was used as a negative control. Purified proteins were used as additional controls (purified PE and BL21).

FIG. 10B shows the toxicity effect of the Pseudomonas exotoxin A (PE) produced according to some embodiments of the present invention on 4T1 cells (mouse breast cancer model). The viability of the cells was determined by MTT assay when four treatment were applied: (1) addition of cell free reaction mixture at 1:20 reaction mixture:growth media ratio; (2) addition of cell free reaction mixture at 1:5 reaction mixture:growth media ratio; (3) addition of particles containing purified proteins (PE/BL21) at 1:20 particles:growth media ratio; (4) addition of particles containing purified proteins (PE/BL21) at 1:5 particles:growth media ratio.

FIG. 11A shows purification of Pseudomonas exotoxin A (PE) from E. coli BL21(DE3) by anion exchange column with NaCl gradient (0-0.5M) for 10 min.

FIG. 11B shows the therapeutic potency of Pseudomonas exotoxin A (PE) on 4T1 and B16 cells. The viability of the cells was determined by MTT assay.

### DESCRIPTION OF SPECIFIC EMBODIMENTS OF THE INVENTION

The present invention, in some embodiments thereof, relates to methods and kits for cell-free RNA or protein synthesis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The invention described herein regards to a short timed, simple and cost effective process for preparing a cell-free protein producing cocktail that is more potent and stable than commercially available cell-free extracts.

TABLE 1
Cost comparison between the classic protocols described in the
literature by Zubay et. al. and Pratt,(supra) two commercially
available systems (NEB, Promega), to the newly invented process.

| Reaction type | Reaction cost (nis/50 μl) | | |
|---|---|---|---|
| The system according to some | 1.82 | | consumables only |
| embodiments of the invention | | {close oversize brace} | |
| Zubay's system ^{7,9} | 5.28 | | |
| S30 T7 High-Yield Protein Expression | 80 | | cost for consumer |
| System, Promega | | {close oversize brace} | |
| PureExpress ®, NEB | 160 | | |

Thus, according to an aspect of the invention there is provided a method for cell-free RNA or protein synthesis, the method comprising synthesizing RNA or a protein of interest in a reaction mixture comprising:

a template DNA encoding the RNA or protein of interest; and

a biological extract of a protease-deficient bacterial cell that has been genetically modified to express a heterologous RNA polymerase, the extract further comprising components necessary for transcription and translation of the protein.

As used herein a “protease-deficient bacterial cell” refers to a bacterial which is down-regulated in at least one protease and thereby allows accumulation of the recombinant protein. Examples of proteases which can be down-regulated (knocked-out) according to some embodiments of the invention include, but are not limited to, Lon and OmpT, hflA, rpoH, tsp.

Bacterial cells are used when the gene to be expressed has been cloned into a vector containing the appropriate prokaryotic regulatory sequences, such as a promoter and ribosome binding site. Prokaryotic E. coli cell-free systems are considered “coupled” because transcription and translation occur simultaneously after the addition of DNA to the extract.

Knocking down gene expression in bacterial cells is done using methods which are well known in the art or by screening for bacterial cells in which protease levels are reduced below a predetermined threashold. Genome editing is an important technology for bacterial cellular engineering, which is commonly conducted by homologous recombination-based procedures, including gene knockout (disruption), and allelic exchange. In addition, recombination-independent approaches have emerged that utilize catalytic RNAs, artificial nucleases, nucleic acid analogs, and peptide nucleic acids. Apart from these methods, which directly modify the genomic structure, an alternative approach is to conditionally modify the gene expression profile at the posttranscriptional level without altering the genomes. This is performed by expressing antisense RNAs to specifically knock down (silence) target mRNAs in vivo. The use of genome editing techniques which make use of artificial/RNA guided/chimeric nucleases such as CRISPR/Cas, TALEN and ZFN are also contemplated herein. Methods of knocking down gene expression in bacterial cells are reviewed in Nakashima and Miyazaki 2014 Int. J. Mol. Sci. 2014 15:2773-2793.

Examples of bacterial cells which can be used in accordance with the present teachings, include but are not limited to E. coli, BL21(DE3)- and E. coli BL21 (DE3)pLysS.

Examples of bacterial strains which can be used along with the present teachings include but are not limited to BL21(DE3) (Novagen), CodonPlusRIL, BL21-SI (Invitrogen) and variants thereof.

Following is a list of bacterial strains which can be used along the teachings of the present invention.

When necessary the bacterial strain may be transformed to express the heterologous RNA polymerase and further modified to be a protease-deficient bacteria.

As used herein “heterologous RNA polymerase” refers to an RNA polymerase that is not naturally expressed in the protease deficient bacterial cell, or which is expressed at a reduced level in the bacterial cell.

According to an embodiment of the invention, the RNA polymerase is an RNA-dependent RNA polymerase. In such a case the template is not a DNA template but rather an RNA template.

According to an embodiment of the invention, the RNA polymerase is a DNA-dependent RNA polymerase.

According to an embodiment of the invention, the DNA-dependent RNA polymerase is a phage RNA polymerase.

According to an embodiment of the invention, the phage RNA polymerase is a T7 or SP6 RNA polymerase.

According to an embodiment of the invention, the T7 is encoded from pAR1219.

Examples of RNA polymerases which can be used along with the present teachings include, but are not limited to SP6, T7 and T3 RNA polymerase. According to a specific embodiment, the RNA polymerase is a T7 RNA polymerase. The polymerase is selected according to the transcriptional control in the template DNA (or vise a versa). A number of vectors containing the SP6, T7 and T3 RNA polymerase promoters are commercially available and are widely used for cloning DNA.

According to an embodiment of the invention, the protease-deficient bacterial cells are selected from the group consisting of BL21(DE3), CodonPlus RIL and variants thereof. Typically, these cells are modified to inducibly express T7 RNA polymerase under control of an inducible promoter e.g., lacUV5 promoter (inducible by addition of IPTG) after incorporation of PAR1219 plasmid, T7 RNA polymerase is produced in excess (over-expression) by T7 RNA polymerase that is expressed from the chromosome.

According to a specific embodiment, the protease deficient bacterial cell is also RNase deficient.

TABLE 2
Examples of bacterial cells which can be used along with the present
teachings or which can be used as a basis for additional manipulations (e.g.,
expression of heterologous RNA polymerase or knocking down the protease, as
described herein)

| Bacterial | | | Growth |
|---|---|---|---|
| Strain | Company | Features | Requirements |
| BL21 (DE3) | Novagen | DE3 lysogen contains T7 polymerase upon IPTG | There is no tight |
| | | induction. This strain is defficient of Ion and omp-t | control over the |
| | | proteases and is therefore suitable for expression | expression, thus |
| | | of non-toxic genes. | addition of 1% |
| | | | glucose to the |
| | | | growth medium |
| | | | should be |
| | | | considered. (Note: |
| | | | 1. This |
| | | | significantly |
| | | | attenuates growth |
| | | | rates. 2. Higher |
| | | | concentrations of |
| | | | IPTG should be |
| | | | used for |
| | | | induction.) |
| BL21 (DE3)- | Novagen | DE3 lysogen expresses T7 polymerase upon IPTG | Chloramphenicol |
| pLysS | | induction. The pLysS plasmid produces T7 | 34 ug/ml |
| | | lysozyme to reduce basal level expression of the | |
| | | gene of interest. Thus it is suitable for expression | |
| | | of toxic genes. | |
| | | [pLysS contains the p15A origin. This origin | |
| | | allows pLysS to be compatible with plasmids | |
| | | containing the ColE1 or pMB1 origin (i.e. pUC- | |
| | | or pBR322-derived plasmids).] | |
| BL21 Star- | Invitrogen | This strain is mutated in Rnase E, thus Rnase | Chloramphenicol |
| pLysS | | degradation is reduced, and protein expression | 34 ug/ml |
| | | may be increased. The pLysS presence allows tight | |
| | | control of expression. Use this strain for both | |
| | | soluble and insoluble protein production. | |
| BL21-SI | Invitrogen | T7 polymerase in the DE3 is under control of the | LBON medium |
| | | salt-inducible proU promoter. Induction of protein | (LB w/o NaCl), |
| | | production at 0.1-0.5M of NaCl. Helps protein | 30° C. |
| | | solubility. | Expression |
| | | | induction by |
| | | | NaCl, if |
| | | | controlled by a T7 |
| | | | promoter. |
| | | | Otherwise-IPTG. |
| BL21-AI | Invitrogen | BL21-AI was constructed by inserting a | Tetracyclin |
| | | chromosomal copy of the T7 RNA polymerase | 12.5 ug/ml |
| | | gene under the tight control of the arabinose- | Expression |
| | | inducible araBAD promoter. | induction by |
| | | | arabinose. (If |
| | | | using pET |
| | | | vectors, IPTG is |
| | | | also required!) |
| | | | Glucose will |
| | | | repress this |
| | | | induction. |
| Tuner | Novagen | Contains a mutation in the lac permease (lacZY) | None |
| | | gene. This enables adjustable levels of protein | |
| | | expression throughout all cells in a culture. The lac | |
| | | permease (lacY) mutation allows uniform entry of | |
| | | IPTG into all cells in the population, which | |
| | | produces a concentration-dependent, homogeneous | |
| | | level of induction. By adjusting the concentration | |
| | | of IPTG, expression can be regulated from very | |
| | | low levels up to the robust, fully induced levels | |
| | | commonly associated with pET vectors. Lower | |
| | | level expression may enhance the solubility and | |
| | | activity of difficult target proteins. | |
| Tuner pLysS | Novagen | Contains the pLysS plasmid (tighter control over | Chloramphenicol |
| | | expression) in addition to the lac permease | 34 ug/ml |
| | | mutation. | |
| Origami | Novagen | Origami host strains are K-12 derivatives that have | Kanamycin |
| | | mutations in both the thioredoxin reductase (trxB) | 15 ug/ml |
| | | and glutathione reductase (gor) genes, which | Tetracyclin |
| | | greatly enhances disulfide bond formation in the | 12.5 ug/ml |
| | | cytoplasm. | |
| Origami B | Novagen | Origami B host strains carry the same trxB/gor | Kanamycin |
| | | mutations as the original Origami strains, except | 15 ug/ml |
| | | that they are derived from a lacZY mutant of | Tetracyclin |
| | | BL21. Thus the Origami B strains combine the | 12.5 ug/ml |
| | | desirable characteristics of BL21, Tuner&amp;trade; | |
| | | and Origami hosts in one strain background. | |
| Origami B | Novagen | | Kanamycin |
| pLysS | | | 15 ug/ml |
| | | | Tetracyclin |
| | | | 12.5 ug/ml |
| | | | Chloramphenicol |
| | | | 34 ug/ml |
| Rosetta | Novagen | Rosetta host strains are BL21 lacZY | Chloramphenicol |
| | | (Tuner&amp;trade;) derivatives designed to enhance | 34 ug/ml |
| | | the expression of eukaryotic proteins that contain | |
| | | codons rarely used in E. coli. These strains | |
| | | supply | |
| | | tRNAs for the codons AUA, AGG, AGA, CUA, | |
| | | CCC, GGA on a compatible chloramphenicol | |
| | | resistant | |
| | | plasmid. The tRNA genes are driven by their | |
| | | native promoters. | |
| Rosetta pLysS | Novagen | In Rosetta(DE3)pLysS the rare tRNA genes are | Chloramphenicol |
| | | present on the same plasmids that carry the T7 | 34 ug/ml |
| | | lysozyme. | |
| Rosetta-gami- | Novagen | Rosetta-gami host strains are Origami derivatives | Kanamycin |
| pLysS | | that combine the enhanced disulfide bond | 15 ug/ml |
| | | formation resulting from trxB/gor mutations with | Tetracyclin |
| | | enhanced expression of eukaryotic proteins | 12.5 ug/ml |
| | | that contain codons rarely used in E. coli. These | Chloramphenicol |
| | | strains supply tRNAs for AGG, AGA, AUA, | 34 ug/ml |
| | | CUA, CCC, GGA on a compatible | |
| | | chloramphenicolresistant plasmid. The tRNA | |
| | | genes are driven by their native promoters. In | |
| | | Rosettagami(DE3)pLysS the rare tRNA genes are | |
| | | present on the same plasmids that carry the T7 | |
| | | lysozyme. | |
| BL21 | Stratagene | BL21-CodonPlus-RIL chemically competent cells | Tetracyclin |
| CodonPlus | | carry extra copies of the argU, ileY, and leuW | 12.5 ug/ml? |
| | | tRNA genes. The tRNAs encoded by these genes | Chloramphenicol |
| | | recognize the AGA/AGG (arginine), AUA | 34 ug/ml? |
| | | (isoleucine), and CUA (leucine) codons, | |
| | | respectively. | |
| AD494 | Novagen | AD494 strains are thioredoxin reductase (trxB) | Kanamycin |
| | | mutants of the K12 strain that enable disulfide | 15 ug/ml |
| | | bond formation in the cytoplasm, providing the | |
| | | potential to produce properly folded active | |
| | | proteins. | |
| BL21trxB | Novagen | BL21trxB strains possess the same thioredoxin | Kanamycin |
| | | reductase mutation (trxB) as the AD494 strains in | 15 ug/ml |
| | | the protease deficient BL21 background. The trxB | |
| | | mutation enables cytoplasmic disulfide bond | |
| | | formation. | |
| HMS174 | Novagen | HMS174 strains provide the recA mutation in a K- | None |
| | | 12 background. Like BLR, these strains may | |
| | | stabilize certain target genes whose products may | |
| | | cause the loss of the DE3 prophage. | |
| NovaBlue(DE3) | Novagen | NovaBlue(DE3) is a K-12 strain ideally suited as | Tetracyclin |
| | | an initial cloning and expression host due to its | 12.5 ug/ml |
| | | high transformation efficiency and recA endA | |
| | | mutations, which result in high yields of excellent | |
| | | quality plasmid DNA. It olso contains DE3 for T7 | |
| | | polymerase expression. Ttight regulation of | |
| | | expression is conferred due to the presence of IacI^{q} | |
| | | repressor encoded by the F episome. | |
| BLR | Novagen | BLR is a recA- derivative of BL21 that improves | Tetracyclin |
| | | plasmid monomer yields and may help stabilize | 12.5 ug/ml |
| | | target plasmids containing repetitive sequences or | |
| | | whose products may cause the loss of the DE3 | |
| | | prophage. | |
| C41(DE3) | Lucigen | Effective in expressing toxic and membrane | None |
| | | proteins from all classes of organisms,including | Protocols |
| | | viruses,eubacteria,archaea,yeasts,plants,insects,and | |
| | | mammals. | |
| | | The strain C41(DE3)was derived from BL21(DE3) | |
| | | [E.coli F ompT hsdS B (r B-m B-) gal dcm | |
| | | (DE3A.This strain has at least one uncharacterized | |
| | | mutation that prevents cell death associated with | |
| | | expression of many toxic recombinant proteins. | |
| C43(DE3) | Lucigen | Effective in expressing toxic and membrane | None |
| | | proteins from all classes of organisms,including | Protocols |
| | | viruses,eubacteria,archaea,yeasts,plants,insects,and | |
| | | mammals. | |
| Lemo21(DE3) | NEB | Lemo21(DE3) offers the host features of | Chloramphenicol |
| (Site) | | BL21(DE3) while also allowing for tunable | L-rhamnose |
| | | expression of difficult clones. Tunable expression | (different |
| | | is achieved by varying the level of lysozyme | concentratins), see |
| | | (lysY), the natural inhibitor of T7 RNA | protocol |
| | | polymerase. The level of lysozyme is modulated | |
| | | by adding L-rhamnose to the expression culture at | |
| | | levels from zero to 2000 μM. When Lemo21(DE3) | |
| | | is grown without rhamnose, the strain performs the | |
| | | same as a pLysS containing strain. However, | |
| | | optional addition of rhamnose tunes the expression | |
| | | of the protein of interest. For difficult soluble | |
| | | proteins, tuning the expression level may also | |
| | | result in more soluble, properly folded protein. | |
| SHuffle T7 | NEB | E. coli K12 cells engineered to form proteins | Protocol |
| (Site) | | containing disulfide bonds in the cytoplasm. | |
| | | Suitable for T7 promoter driven protein | |
| | | expression. | |
| | | Expresses constitutively a chromosomal copy of | |
| | | the disulfide bond isomerase DsbC. DsbC | |
| | | promotes the correction of mis-oxidized proteins | |
| | | into their correct form. The cytoplasmic DsbC is | |
| | | also a chaperone that can assist n the folding of | |
| | | proteins that do not require disulfide bonds. | |
| ArcticExpress | Agilent | ArcticExpress Competent Cells are engineered to | For protein |
| and | Technologies | address the common bacterial gene expression | expression: |
| ArcticExpress | | hurdle of protein insolubility. These cells are | Gentamycin |
| (DE3) | | derived from the high-performance Stratagene | 20 ug/mL |
| (Site) | | BL21-Gold competent cells, enabling efficient | For |
| | | high-level expression of heterologous proteins in | transformation: |
| | | Escherichia coli. | no need for |
| | | | antibiotic other |
| | | | than the one of the |
| | | | transformed |
| | | | plasmid. |
| | | | For making |
| | | | compotent cells: |
| | | | Gentamycin |
| | | | 20 ug/mL, |
| | | | Tetracycline |
| | | | 10 ug/mL |

According to an embodiment of the invention the biological extract of a protease-deficient bacterial cell that has been genetically modified to express a heterologous RNA polymerase is produced as follows.

(a) growing a culture of protease-deficient bacterial cells that have been genetically modified to express a heterologous RNA polymerase under conditions which allow expression of the RNA polymerase;

(b) disrupting the protease-deficient bacterial cells to obtain a cell preparation which comprises genomic DNA and broken cells; the disrupting being under conditions which retain functionality of components necessary for transcription and translation of the protein;

(c) removing the genomic DNA. At this stage other cell debris are also removed (e.g., membranes); and

(d) freezing the cell preparation following the removing.

Thus, the bacterial cell is transformed to express a heterologous RNA polymerase. The expression of the polymerase is typically effected in an induced expression manner such as by the use of IPTG (e.g., at OD₆₀₀≧1). Other modes of expression induction are also known in the art e.g., salt induction, tetracycline, arabinose and theophyline.

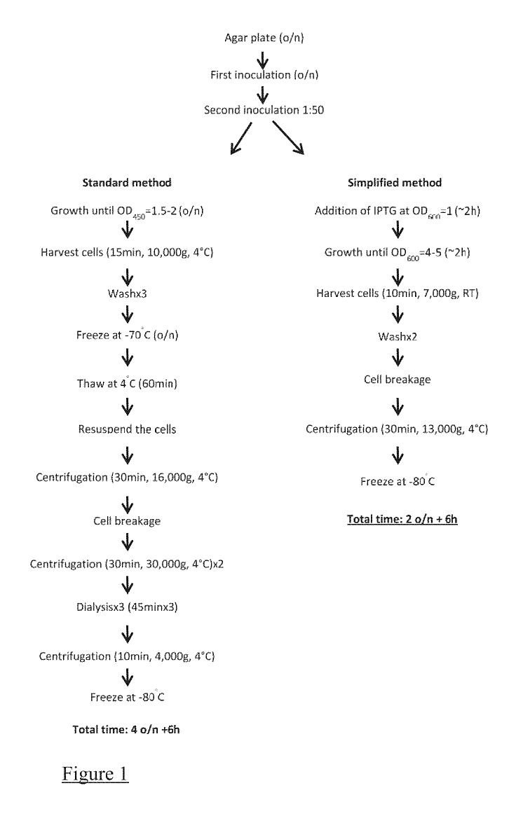

Once the culture reaches a pre-determined optical density (OD) (e.g., OD₆₀₀≧5, e.g., 4-5) the cells are harvested e.g., by centrifugation and typically washed to remove cell debris. According to a specific embodiment, two washing steps are employed (see FIG. 1).

The optical density for cell induction and harvesting depends on the medium used for culturing and it is well within the capabilities of the skilled artisan to determine same.

According to a specific embodiment, disrupting is effected using a homogenizer. According to a specific embodiment, disrupting is effected using a French press.

Removal of genomic DNA is effected typically by centrifugation though other methods are also contemplated. According to a specific embodiment, centrifugation is effected at 13,000-20,000 g or less. According to a specific embodiment centrifugation is effected once.

According to a specific embodiment, the method doesn't include dialyzing the cell preparation following the disrupting, which minimizes the production time significantly.

According to an additional or alternative embodiment the method does not include freezing and thawing the bacterial cells following step (a) and prior to step (b).

According to a specific embodiment the method does not comprise adding a protease inhibitor to the cell preparation. Hence the extract, according to a specific embodiment does not comprise externally added protease inhibitors.

The preparation can be used fresh or after freezing, typically at −20° C. or −80° C.

An exemplary embodiment for the method of producing the bacterial extract of the invention is provided in FIG. 1 and Figure.

As mentioned the present teachings are directed to the coupled production of RNA and protein. Though, in certain embodiments a non-coding RNA can be expressed. The term “protein” or “protein of interest” refers to a peptide or polypeptide having more than about 5 amino acids. The protein may be homologous to, or heterologous, i.e., foreign, to the bacteria from which the bacterial cell-free extract is derived, such as a human protein, viral protein, yeast protein, insect protein, bacterial protein, etc. produced in the bacterial cell-free extract.

According to a specific embodiment, the protein is a membranal protein, a secreted protein or an intracellular protein.

Heterologous production of proteins is widely employed in research and industrial settings, for example, for production of therapeutics, vaccines, diagnostics, biofuels, and many other applications of interest.

Exemplary enzyme classes that can be synthesized according to the present teachings include, but are not limited to: EC1 (oxidoreductases), EC2 (transferases), EC3 (hydrolases), EC4 (lyases), EC5 (isomerases) and EC6 (ligases).

Additional proteins of interest include, but are not limited to antibodies, lysosomal enzymes, protelolytic enzymes, lipases, toxins.

Proteins or peptides produced by the the present teachings can include, but are not limited to cytokines, chemokines, lymphokines, ligands, receptors, hormones, enzymes, antibodies and antibody fragments, and growth factors. Non-limiting examples of receptors include TNF type I receptor, IL-1 receptor type II, IL-1 receptor antagonist, IL-4 receptor and any chemically or genetically modified soluble receptors. Examples of enzymes include acetlycholinesterase, lactase, activated protein C, factor VII, collagenase (e.g., marketed by Advance Biofactures Corporation under the name Santyl); agalsidase-beta (e.g., marketed by Genzyme under the name Fabrazyme); dornase-alpha (e.g., marketed by Genentech under the name Pulmozyme); alteplase (e.g., marketed by Genentech under the name Activase); pegylated-asparaginase (e.g., marketed by Enzon under the name Oncaspar); asparaginase (e.g., marketed by Merck under the name Elspar); and imiglucerase (e.g., marketed by Genzyme under the name Ceredase). Examples of specific polypeptides or proteins include, but are not limited to granulocyte macrophage colony stimulating factor (GM-CSF), granulocyte colony stimulating factor (G-CSF), macrophage colony stimulating factor (M-CSF), colony stimulating factor (CSF), interferon beta (IFN-beta), interferon gamma (IFNgamma), interferon gamma inducing factor I (IGIF), transforming growth factor beta (IGF-beta), RANTES (regulated upon activation, normal T-cell expressed and presumably secreted), macrophage inflammatory proteins (e.g., MIP-1-alpha and MIP-1-beta), Leishmnania elongation initiating factor (LEIF), platelet derived growth factor (PDGF), tumor necrosis factor (TNF), growth factors, e.g., epidermal growth factor (EGF), vascular endothelial growth factor (VEGF), fibroblast growth factor, (FGF), nerve growth factor (NGF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), TNF alpha type II receptor, erythropoietin (EPO), insulin and soluble glycoproteins e.g., gp120 and gp160 glycoproteins. The gp120 glycoprotein is a human immunodeficiency virus (WIV) envelope protein, and the gp160 glycoprotein is a known precursor to the gp120 glycoprotein. Other examples include secretin, nesiritide (human B-type natriuretic peptide (hBNP)) and GYP-I.

Other products may include GPCRs, including, but not limited to Class A Rhodopsin like receptors such as Muscatinic (Muse.) acetylcholine Vertebrate type 1, Musc. acetylcholine Vertebrate type 2, Musc. acetylcholine Vertebrate type 3, Musc. acetylcholine Vertebrate type 4; Adrenoceptors (Alpha Adrenoceptors type 1, Alpha Adrenoceptors type 2, Beta Adrenoceptors type 1, Beta Adrenoceptors type 2, Beta Adrenoceptors type 3, Dopamine Vertebrate type 1, Dopamine Vertebrate type 2, Dopamine Vertebrate type 3, Dopamine Vertebrate type 4, Histamine type 1, Histamine type 2, Histamine type 3, Histamine type 4, Serotonin type 1, Serotonin type 2, Serotonin type 3, Serotonin type 4, Serotonin type 5, Serotonin type 6, Serotonin type 7, Serotonin type 8, other Serotonin types, Trace amine, Angiotensin type 1, Angiotensin type 2, Bombesin, Bradykffin, C5a anaphylatoxin, Finet-leu-phe, APJ like, Interleukin-8 type A, Interleukin-8 type B, Interleukin-8 type others, C-C Chemokine type 1 through type 11 and other types, C-X-C Chemokine (types 2 through 6 and others), C-X3-C Chemokine, Cholecystokinin CCK, CCK type A, CCK type B, CCK others, Endothelin, Melanocortin (Melanocyte stimulating hormone, Adrenocorticotropic hormone, Melanocortin hormone), Duffy antigen, Prolactin-releasing peptide (GPR10), Neuropeptide Y (type 1 through 7), Neuropeptide Y, Neuropeptide Y other, Neurotensin, Opioid (type D, K, M, X), Somatostatin (type 1 through 5), Tachykinin (Substance P(NK1), Substance K (NK2), Neuromedin K (NK3), Tachykinin like 1, Tachykinin like 2, Vasopressin/vasotocin (type 1 through 2), Vasotocin, Oxytocin/mesotocin, Conopressin, Galanin like, Proteinase-activated like, Orexin & neuropeptides FF, QRFP, Chemokine receptor-like, Neuromedin U like (Neuromedin U, PRXamide), hormone protein (Follicle stimulating hormone, Lutropin-choriogonadotropic hormone, Thyrotropin, Gonadotropin type I, Gonadotropin type II), (Rhod)opsin, Rhodopsin Vertebrate (types 1-5), Rhodopsin Vertebrate type 5, Rhodopsin Arthropod, Rhodopsin Arthropod type 1, Rhodopsin Arthropod type 2, Rhodopsin Arthropod type 3, Rhodopsin Mollusc, Rhodopsin, Olfactory (Olfactory 11 fam 1 through 13), Prostaglandin (prostaglandin E2 subtype EP 1, Prostaglandin E2/D2 subtype EP2, prostaglandin E2 subtype EP3, Prostaglandin E2 subtype EP4, Prostaglandin F2-alpha, Prostacyclin, Thromboxane, Adenosine type 1 through 3, Purinoceptors, Purinoceptor P2RY1-4,6,11 GPR91, Purinoceptor P2RY5,8,9,10 GPR35,92,174, Purinoceptor P2RY12-14 GPR87 (JDP-Glucose), Cannabinoid, Platelet activating factor, Gonadotropin-releasing hormone, Gonadotropin-releasing hormone type I, Gonadotropin-releasing hormone type II, Adipokinetic hormone like, Corazonin, Thyrotropin-releasing hormone & Secretagogue, Thyrotropin-releasing hormone, Growth hormone secretagogue, Growth hormone secretagogue like, Ecdysis-triggering hormone (ETHR), Melatonin, Lysosphingolipid & LPA (EDG), Sphingosine 1-phosphate Edg-1, Lysophosphatidic acid Edg-2, Sphingosine 1-phosphate Edg-3, Lysophosphatidic acid Edg4, Sphingosine 1-phosphate Edg-5, Sphingosine 1-phosphate Edg-6, Lysophosphatidic acid Edg-7, Sphingosine 1-phosphate Edg-8, Edg Other Leukotriene B4 receptor, Leukotriene B4 receptor BLT1, Leukotriene B4 receptor BLT2, Class A Orphan/other, Putative neurotransmitters, SREB, Mas proto-oncogene & Mas-related (MRGs), GPR45 like, Cysteinyl leukotriene, G-protein coupled bile acid receptor, Free fatty acid receptor (GP40, GP41, GP43), Class B Secretin like, Calcitonin, Corticotropin releasing factor, Gastric inhibitory peptide, Glucagon, Growth hormone-releasing hormone, Parathyroid hormone, PACAP, Secretin, Vasoactive intestinal polypeptide, Latrophilin, Latrophilin type 1, Latrophilin type 2, Latrophilin type 3, ETL receptors, Brain-specific angiogenesis inhibitor (BAI), Methuselah-like proteins (MTH), Cadherin EGF LAG (CELSR), Very large G-protein coupled receptor, Class C Metabotropic glutamate/pheromone, Metabotropic glutamate group I through III, Calcium-sensing like, Extracellular calcium-sensing, Pheromone, calcium-sensing like other, Putative pheromone receptors, GABA-B, GABA-B subtype 1, GABA-B subtype 2, GABA-B like, Orphan GPRC5, Orphan GPCR6, Bride of sevenless proteins (BOSS), Taste receptors (TiR), Class D Fungal pheromone, Fungal pheromone A-Factor like (STE2,STE3), Fungal pheromone B like (BAR,BBR,RCB,PRA), Class E cAMP receptors, Ocular albinism proteins, Frizzled/Smoothened family, frizzled Group A (Fz 1&2&4&5&7-9), frizzled Group B (Fz 3 & 6), fizzled Group C (other), Vomeronasal receptors, Nematode chemoreceptors, Insect odorant receptors, and Class Z Archaeal/bacterial/fungal opsins.

Bioactive peptides may also be produced by the present invention. Examples include: BOTOX, Myobloc, Neurobloc, Dysport (or other serotypes of botulinum neurotoxins), alglucosidase alfa, daptomycin, YH-16, choriogonadotropin alfa, filgrastim, cetrorelix, interleukin-2, aldesleukin, teceleulin, denileukin diftitox, interferon alfa-n3 (injection), interferon alfa-nl, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alfa, collagenase, carperitide, recombinant human epidermal growth factor (topical gel, wound healing), DWP401, darbepoetin alfa, epoetin omega, epoetin beta, epoetin alfa, desirudin, lepirudin, bivalirudin, nonacog alpha, Mononine, eptacog alfa (activated), recombinant Factor VIII+VWF, Recombinate, recombinant Factor VIII, Factor VIII (recombinant), Alphnmate, octocog alfa, Factor VIII, palifermin, Indikinase, tenecteplase, alteplase, pamiteplase, reteplase, nateplase, monteplase, follitropin alfa, rFSH, hpFSH, micafungin, pegfilgrastim, lenograstim, nartograstim, sermorelin, glucagon, exenatide, pramlintide, iniglucerase, galsulfase, Leucotropin, molgramostim, triptorelin acetate, histrelin (subcutaneous implant, Hydron), deslorelin, histrelin, nafarelin, leuprolide sustained release depot (ATRIGEL), leuprolide implant (DUROS), goserelin, somatropin, Eutropin, KP-102 program, somatropin, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alfa, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alfa, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alfa-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMlD, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, AOD-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10(autoimmune iseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alfa-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague F1V vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In certain embodiments, the protein is an enzyme or biologically active fragments thereof. Suitable enzymes include but are not limited to: oxidoreductases, transferases, hydrolases, lyases, isomerases, and ligases. In certain embodiments, the heterologously produced protein is an enzyme of Enzyme Commission (EC) class 1, for example an enzyme from any of EC 1.1 through 1.21, or 1.97. The enzyme can also be an enzyme from EC class 2, 3, 4, 5, or 6. For example, the enzyme can be selected from any of EC 2.1 through 2.9, EC 3.1 to 3.13, EC 4.1 to 4.6, EC 4.99, EC 5.1 to 5.11, EC 5.99, or EC 6.1-6.6.

As used herein, the term “antibody” refers to a substantially intact antibody molecule.

As used herein, the phrase “antibody fragment” refers to a functional fragment of an antibody (such as Fab, F(ab′)2, Fv or single domain molecules such as VH and VL) that is capable of binding to an epitope of an antigen.

According to one embodiment, the polypeptides are derived from a mammalian species for example human polypeptides.

According to a specific embodiment, the protein is selected from the group consisting of sfGFP, P50, tyrosinase and Pseudomonas exotoxin.

As mentioned, the present teachings may also be employed towards the production of RNA only. The use of non-coding RNA in research, as well as in the clinic, agriculture, industrial applications (e.g., dairy production) has gained a lot of attention in the last decades.

According to a specific embodiment, the RNA is an RNA silencing agent.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs (or miR mimics) and shRNAs.

In one embodiment, the RNA silencing agent is capable of inducing RNA interference.

In another embodiment, the RNA silencing agent is capable of mediating translational repression.

The RNA may be of 10-1000 bases long.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

miRNA and miRNA Mimics—

According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

The extract may also comprise mRNA encoding the heterologous RNA polymerase and the plasmid comprising the nucleic acid sequence expressing the heterologous RNA polymerase.

According to a specific embodiment the entire RNA polymerase activity for heterologous protein expression in the reaction mixture is substantially attributed to the expression, presence or level of the heterologous RNA polymerase.

According to an alternative embodiment, the reaction mixture further comprises a heterologous RNA polymerase which is exogenously added to the mixture as a purified protein (free of host cell contaminants).

The bacterial host cell is protease free for improving expression of recombinant proteins and may also be RNAse free.

According to a specific embodiment, the protease deficient bacterial cell is also RNase deficient.

Once the cell extract is obtained, it may be encapsulated in lipid particles. This may be used in clinical applications to ensure in vivo production of proteins/mRNA in a localized/targeted manner. To this end (e.g., in systemic applications) the particle may be modified to present targeting moieties. These targeting moieties include ligands, such as oligosaccharides, peptides, proteins and vitamins. Most targeting moieties are typically focused on antibody conjugation since procedures for producing monoclonal antibodies against a tissue target of interest are well established.

As used herein, “vesicle” refers to nano to micro structures (e.g., 100 nm-5 μm) which are not biological cells.

The particle may be a synthetic carrier having an internal cavity which is capable of being loadable with (e.g., encapsulating) the extract. The particle may be either polymeric or non-polymeric preparations.

Exemplary particles that may be used according to this aspect of the present invention include, but are not limited to polymeric particles, microcapsules, liposomes, microspheres, microemulsions, nanoparticles, nanocapsules, nano-spheres, nano-liposomes, nano-emulsions and nanotubes.

According to a particular embodiment, the particles are nanoparticles.

As used herein, the term “nanoparticle” refers to a particle or particles having an intermediate size between individual atoms and macroscopic bulk solids. Generally, nanoparticle has a characteristic size (e.g., diameter for generally spherical nanoparticles, or length for generally elongated nanoparticles) in the sub-micrometer range, e.g., from about 100 nm to about 5000 nm, from about 100 nm to about 500 nm, or from about 100 nm to about 300 nm, or 300 nm-1000 nm. The nanoparticles may be of any shape, including, without limitation, elongated particle shapes, such as nanowires, or irregular shapes, in addition to more regular shapes, such as generally spherical, hexagonal and cubic nanoparticles. According to one embodiment, the nanoparticles are generally spherical. The size will generally be selected according to the intended use. Thus for localized treatment such as for the treatment of fibrosis (e.g., using particles expressing a proteolytic enzyme e.g., collagenase, the particles may be of an average diameter of 500 nm-5000 nm, on the other hand for the treatment of cancer e.g., using an anti-proliferative agent the particle used may be smaller e.g., 100-300 nm, such particles are also used in the treatment of liver indications).

The particles of this aspect of the present invention may have a charged surface (i.e., positively charged or negatively charged) or a neutral surface.

Agents which are used to fabricate the particles may be selected according to the desired charge required on the outer surface of the particles.

Thus, for example if a negatively charged surface is desired, the particles may be fabricated from negatively charged lipids (i.e. anionic phospholipids) such as described herein below.

When a positively charged surface is desired, the particles may be fabricated from positively charged lipids (i.e. cationic phospholipids), such as described herein below.

As mentioned, non charged particles are also contemplated by the present invention. Such particles may be fabricated from neutral lipids such as phosphatidylethanolamine or dioleilphosphatidylethanolamine (DOPE).

It will be appreciated that combinations of different lipids may be used to fabricate the particles of the present invention, including a mixture of more than one cationic lipid, a mixture of more than one anionic lipid, a mixture of more than one neutral lipid, a mixture of at least one cationic lipid and at least one anionic lipid, a mixture of at least one cationic lipid and at least one neutral lipid, a mixture of at least one anionic lipid and at least one neutral lipid and additional combinations of the above. In addition, polymer-lipid based formulations may be used.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie-polyglycolic acid′ polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The particles may also include other components. Examples of such other components includes, without being limited thereto, fatty alcohols, fatty acids, and/or cholesterol esters or any other pharmaceutically acceptable excipients which may affect the surface charge, the membrane fluidity and assist in the incorporation of the biologically active lipid into the lipid assembly. Examples of sterols include cholesterol, cholesterol hemisuccinate, cholesterol sulfate, or any other derivatives of cholesterol. Preferred lipid assemblies according the invention include either those which form a micelle (typically when the assembly is absent from a lipid matrix) or those which form a liposome (typically, when a lipid matrix is present).

In a specific embodiment, the particle is a liposome. As used herein and as recognized in the art, liposomes include any synthetic (i.e., not naturally occurring) structure composed of lipid, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43].

The liposomes may be unilamellar or may be multilamellar. Unilamellar liposomes may be preferred in some instances as they represent a larger surface area per lipid mass. Suitable liposomes in accordance with the invention are preferably non-toxic. The liposomes may be fabricated from a single phospholipid or mixtures of phospholipids. The liposomes may also comprise other lipid materials such as cholesterol. For fabricating liposomes with a negative electrical surface potential, acidic phospho- or sphingo- or other synthetic-lipids may be used. Preferably, the lipids have a high partition coefficient into lipid bilayers and a low desorption rate from the lipid assembly. Exemplary phospholipids that may be used for fabricating liposomes with a negative electrical surface potential include, but are not limited to phosphatidylserine, phosphatidic acid, phosphatidylcholine and phosphatidyl glycerol.

Other negatively charged lipids which are not liposome forming lipids that may be used are sphingolipids such as cerebroside sulfate, and various gangliosides.

According to a specific embodiment, the liposome is composed of cholesterol and DMPC as described in the Examples section which follows.

The lipid phase of the liposome may comprise a physiologically acceptable liposome forming lipid or a combination of physiologically acceptable liposome forming lipids. Liposome-forming lipids are typically those having a glycerol backbone wherein at least one of the hydrofoil groups is substituted with an acyl chain, a phosphate group, a combination or derivatives of same and may contain a chemically reactive group (such as an as amine imine, acids ester, aldelhyde or alcohol) at the headgroup. Typically, the acyl chain is between 12 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to one embodiment, the lipid phase comprises phospholipids.

The phospholipids may be a glycerophospholipid. Examples of glycerophospholipid include, without being limited thereto, phosphatidylglycerol (PG) including dimyristoyl phosphatidylglycerol (DMPG); phosphatidylcholine (PC), including egg yolk phosphatidylcholine and dimyristoyl phosphatidylcholine (DMPC), phosphatidic acid (PA), phosphatidylinositol (PI), phosphatidylserine (PS) and sphingomyelin (SM) and derivatives of the same.

Another group of lipid matrix employed according to the invention includes cationic lipids (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge.

Preferably, the head groups of the lipid carries the positive charge. Monocationic lipids may include, for example, 1,2-dimyristoyl-3-trimethylammonium propane (DMTAP) 1,2-dioleyloxy-3-(trimethylanino) propane (DOTAP), N—[-1-(2,3-ditetradecyloxy)propyl]-N,N-dimethyl-N-hydroxyethylammonium bromide (DMRIE), N-[1-(2,3-dioleyloxy)propyl]-N,N-dimethyl-N-hydroxy ethyl-ammonium bromide (DORIE), N-[1-(2,3-dioleyloxy) propyl]; —N,N,N-trimethylammonium chloride (DOTMA); 3; N—(N′,N′-dimethylaminoethane) carbamoly]; cholesterol (DC-Chol), and I dimethyl-dioctadecylammonium (DDAB).

Examples of polycationic lipids include a similar lipoplilic moiety as with the mono cationic lipids, to which spermine or spermidine is attached. These include′ without being limited thereto, N-[2-[[2,5-bis[3-aminopropyl)amino]-1-oxopentyl]amino]ethyl]N,N dimethul-2,3 bis(1-oXo-9-octadecenyl)oXy]; -1 propanaminium (DOSPA), and ceramide carbamoyl spermine (CCS).

The cationic lipids may be used alone, in combination with cholesterol, with neutral phospholipids or other known lipid assembly components. In addition, the cationic lipids may form part of a derivatized phospholipids such as the neutral lipid dioleoylphosphatidyl ethanolamine (DOPE) derivatized with polylysine to form a cationic lipopolymer.

For sizing liposomes, extrusion, homogenization or exposure to ultrasound irradiation may be used, Homogenizers which may be conveniently used include microfluidizers produced by Microfluidics of Boston, Mass. In a typical homogenization procedure, liposomes are recirculated through a standard emulsion homogenizer until selected liposomes sizes are observed. The particle size distribution can be monitored by conventional laser beam particle size discrimination. Extrusion of liposomes through a small-pore polycarbonate membrane or an asymmetric ceramic membrane is an effective method for reducing liposome sizes to a relatively well defined size distribution. Typically, the suspension is cycled through the membrane one or more times until the desired liposome size distribution is achieved. The liposomes may be extruded through successively smaller pore membranes to achieve a gradual reduction in liposome size.

Dehydrated lipids or preassembled lipid vesicles (e.g., 20-100 nM, e.g., 50 nM) may be rehydrated in the presence of the extract and DNA template to form lipid vesicles comprising (encapsulating the extract). Importantly, the encapsulation is effected on ice to prevent transcription/translation which is not within the vesicles.

When using particulated extract (i.e., encapsulated in particles), components necessary for transcription/translation (e.g., amino acids, nucleotides, ATP) may be added to the mixture where they are uptaken by the particles.

As used herein “components necessary for protein transcription and translation” refers to the cellular machinery for each of these activities including tRNAs, ribosomes and transcription factors.

In vitro synthesis or “cell free protein synthesis” refers to the cell-free synthesis of polypeptides in a reaction mix comprising biological extracts and/or defined reagents. The reaction comprises a template for production of the macromolecule, e.g. DNA, mRNA, protein.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. The cell free synthesis reaction may be performed as batch, continuous flow, or semi-continuous flow, as known in the art.

According to a specific embodiment, biological extract is present in the reaction mixture at a concentration of 30% (20-40%) (v/v).

According to an embodiment of the invention, the reaction mixture further comprises amino acids, rNTPs, H₂O, salt and an ATP-regenerating system.

According to an embodiment of the invention, the reaction mixture comprises polyethylene glycol.

According to an embodiment of the invention, the template DNA is a circular DNA.

According to an embodiment of the invention, the template DNA comprises a promoter for the RNA polymerase being operably linked to a nucleic acid sequence encoding the protein.

According to a specific embodiment, the DNA is present in the reaction mixture at a concentration of 1-100 μg/μl e.g., 10 μg/μl.

According to an embodiment of the invention, the salt is selected from the group consisting of potassium, magnesium and ammonium.

For coupled transcription and translation the magnesium concentration of the bacterial cellular lysate must be adjusted by an additional magnesium compound, preferably a salt. Preferred salts include magnesium chloride and magnesium acetate. The addition of a buffering agent can be used in the solution to stabilize the pH, although this is not necessary. For coupling transcription and translation, a sufficient amount of magnesium chloride or acetate is added to the lysate to raise the final magnesium concentration to a level where RNA is transcribed from DNA and RNA translates into protein. This level will vary depending upon the lysate used.

Magnesium is also known to be important for optimizing translation, as it enhances the stability of assembled ribosomes and functions in their binding together during translation. Magnesium also appears to play a role in facilitating polymerase binding. Potassium is important as well for optimizing translation, but unlike the case for magnesium, for coupled transcription and translation the concentration of potassium ions does not need to be altered beyond standard translation preparation levels.

The levels are partially from the endogenous lysate levels, and partially from the additions made in the preparation of the lysate.

According to a specific embodiment, the magnesium concentration is adjusted to within a rather narrow optimal range, thefore the lysate magnesium levels is measured directly through the use of a magnesium assay, prior to the addition of extra magnesium, so that the amount of magnesium in a reaction can be standardized from one batch of lysate to the next. The Lancer “Magnesium Rapid Star Diagnostic Kit” (Oxford Lab Ware Division, Sherwood Medical Co., St. Louis, Mo.), is one such assay which can accurately measure the magnesium levels in biological fluid. Once the magnesium ion concentration for a given batch of lysate is known then additional magnesium, for instance in the form of a concentrated magnesium salt solution, can be added in a known manner to bring the magnesium concentration of the lysate to within the optimal range, or, in the case of a modified lysate preparation to be used as one-half of a reaction mixture, to within twice the optimal range. According to an embodiment of the invention, the reaction mixture comprises 10-20 mM magnesium salt, 40-60 mM potassium salt and 100-200 mM ammonium salt.

Reaction conditions for coupled transcription and translation include the addition of ribonucleotide triphosphates (ATP, GTP, CTP, UTP) and amino acids, for the bacterial lysate to final concentrations of 0.8-1.2 mM each, and 2.5 mM. each respectively. If a radiolabeled amino acid is used in the coupled reaction, such as ³⁵S methionine or ³H leucine, then the corresponding amino acid is left out of the amino acid mix. As the lystae already comprises the RNA polymerase, then no additional polymerase is added to the mixture, The DNA template with the gene to be transcribed/translated is added at a concentration of 10 μg/ml and the reaction volume is adjusted to 50 μl with the addition of water (DNase free RNase free). The reaction is then incubated at 37° C. for 1-2 hours, dependent on the product.

Although potassium is added to the reaction mixture as mentioned above, in contrast to magnesium additional potassium does not greatly increase protein production, but only offers a slight improvement when proper magnesium levels are already present. Potassium salt (e.g., acetate) is added to an optimal final concentration of about 50 mM.

The final concentration of potassium chloride or acetate is also an estimation based on the amount of this component in standard lysate, but it must be recognized that this concentration, as well as the magnesium concentration, can vary slightly due to endogenous components.

Additional components can be added to the lysate as desired for improving the efficiency or stability of the coupled transcription and translation reaction. One common addition to coupled transcription and translation reactions is an amount of a polyamine sufficient to stimulate the efficiency of chain elongation.

Although not necessary, but according to a specific embodiment, for coupled transcription and translation, spermidine may be added to the mixture. Polyamines affect optimal magnesium levels as well, and are known to lower the effective magnesium concentration for translation reactions somewhat. It appears that the polyamines may substitute for magnesium at some level, and thus would play a role in the optimization of magnesium requirements, possibly even permitting some lowering of optimal magnesium levels for coupled transcription and translation.

Optimal magnesium concentrations in the in vitro environment are affected by other conditions and considerations, too. As the ribonucleotide triphosphate concentration goes up, for instance, there is a concomitant increase in the optimal magnesium concentration, as the ribonucleotide triphosphates tend to associate, or chelate, with magnesium in solution.

PEG or other synthetic polymers may be added to increase the viscosity of the reaction mix.

According to an embodiment of the invention, the reaction mixture is as listed in Table 3 below.

According to an embodiment of the invention, the synthesizing is effected in batch, continuous flow, or semi-continuous flow.

According to an embodiment of the invention, the yield of the protein of interest is at least about 600 μg protein/ml of reaction mixture, at least about 800 μg protein/ml of reaction mixture, 1000 μg protein/ml of reaction mixture, or more.

According to an exemplary embodiment, the yield of the protein of interest is 100-1500 μg protein/ml of reaction mixture, 500-1500 μg protein/ml of reaction mixture, 600-1500 μg protein/ml of reaction mixture, 700-1500 μg protein/ml of reaction mixture, 800-1500 μg protein/ml of reaction mixture or 1000-1500 μg protein/ml of reaction mixture.

The amount of protein produced in a coupled in vitro transcription and translation can be measured in various fashions. One method relies on the availability of an assay which measures the activity of the particular protein being translated. An example of an assay for measuring protein activity is the luciferase assay system described in Technical Bulletin 101, Promega Corp., Madison, Wis. These assays measure the amount of functionally active protein produced from the coupled in vitro transcription and translation reaction.

Another method of measuring the amount of protein produced in coupled in vitro transcription and translation reactions is to perform the reactions using a known quantity of radiolabeled amino acid such as ³⁵S methionine or ³H leucine and subsequently measuring the amount of radiolabeled amino acid incorporated into the newly translated protein. For a description of this method see the in vitro Translation Technical Manual, Promega Corp., Madison, Wis. Incorporation assays will measure the amount of radiolabeled amino acids in all proteins produced in an in vitro translation reaction including truncated protein products. It is important to separate the radiolabeled protein on a protein gel, and by autoradiography confirm that the product is the proper size and that secondary protein products have not been produced. The most accurate measure of protein production is to correlate the measure of activity with the measurements of incorporation.

According to a specific embodiment, the extract is used in a crude form supplemented with then necessary components such as described hereinbelow, including but not limited to:

(i) lipid which are preformed as or capable of forming lipid vesicles;
(ii) a control DNA template;
(iii) amino acids;
(iv) rNTPs;

### (v) H₂O;

(vi) salt; and
(vii) an ATP-regenerating system.

The extract described herein can be included as part of a kit for facilitating the set up of cell-free translation reactions. Such a kit improves the convenience to the researcher, as the bacterial lystae lysate comes prepared and ready for use. In addition to the extract (also referred to herein as “lysate”), such a kit can include the components, reagents, including nucleotides, salts, and buffers necessary to perform coupled transcription and translation upon the introduction of a DNA template. The lysate can be standard, or can be of the type where the adjustments to its salt concentrations have already been made during manufacture, or additionally where one or more of the components, reagents or buffers necessary for coupled transcription and translation have been included.

Thus, the extract may be packed in a kit comprising instructions for use or further comprising at least one of:

(i) lipid which are preformed as or capable of forming lipid vesicles;
(ii) a control DNA template;
(iii) amino acids;
(iv) rNTPs;

### (v) H₂O;

(vi) salt; and
(vii) an ATP-regenerating system.According to a specific embodiment, the control DNA template is separately packaged from the extract.

According to a specific embodiment, each of the (i)-(vi) is separately packaged.

According to a specific embodiment, at least two of the (i)-(vi) are separately packaged.

For therapeutic indications the particles comprising the extract and template DNA may be administered to a subject in need thereof per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### A Simple Procedure for Preparing S30 Lysate for In Vitro Transcription and Translation of Proteins

Materials and Methods

Bacterial strains and plasmids: TargeTron® Vector pAR1219 was purchased from Sigma-Aldrich (Rehovot, Israel) and transformed into E. coli BL21 (DE3) F⁻ ompT gal dcm ion hsdS_{B}(r_{B}_{^{−}}m_{B}_{^{−}}) λ(DE3 [lacI lacUV5-T7 gene 1 ind1 sam7 nin5) (Novagen®, Merck KgaA, Darmstadt, Germany) competent cells via electroporation using MicroPulser electroporator (Biorad, Hercules, Calif., USA). Plasmid pAR1219 expresses T7 RNA Polymerase under control of the isopropyl β-D-1-thiogalactopyranoside (IPTG) inducible lacUV5 promoter.

Preparation of T7-S30 Lysate

E. coli BL21(DE3)/pAR1219 glycerol stock (−80° C.) was streaked on Luria Bertani (LB)¹⁴ plate solidified with 1.5% Bacto agar, (Acumedia, Neogen corporation, MI, USA) supplemented with ampicillin at 50 μg ml⁻¹ (LB-amp50) to maintain the plasmids. A single colony was used to inoculate fresh LB-amp50 media, the culture was grown over-night (o/n) at 37° C. with shaking at 250 rpm on a TU-400 incubator shaker (Orbital shaker incubator, MRC, Holon, Israel) and used as a starter to inoculate fresh Terrific Broth media¹⁵ (supplemented with 50 μg ml⁻¹ ampicillin) the following day at 1:50 starter:medium ratio. The culture was grown at 37° C. to OD₆₀₀≈1, upon which 0.4 mM of IPTG (Inalco S.P.A., Milano, Italy) were added. The culture was further grown at 37° C. until it reached OD₆₀₀=4 and then centrifuged at 7,000×g for 10 min at 4° C. using F-14 carbon fiber rotor Multifuge 3XR Plus Heraeus centrifuge (Thermo Fisher Scientific, Waltham, Mass., USA). The pellet was re-suspended in the same volume (1:1 v/v) of S30 lysate buffer containing: Tris-acetate 10 mM at pH=7.4 (Sigma-Aldrich), magnesium acetate 14 mM (Merck KGaA, Darmstadt, Germany), potassium acetate 60 mM (Alfa-Aeser, Ward Hill, Mass. USA), dithiothreitol (DTT) 1 mM (Sigma-Aldrich) and 2-mercaptoethanol 0.5 ml/1 lit (Sigma-Aldrich). Afterwards, the suspension was centrifuged again in the same conditions and resuspended in S30 lysate buffer according to the equation: pellet originated from 1 lit culture at OD₆₀₀=5 should be resuspended with 15 ml S30 lysate buffer. Then the cells were broken by one pass through ice cold emulsiFlex-C3 high pressure homogenizer, (Avestin, Mannheim, Germany) at working pressure of 15,000 psi and air pressure of 4 bar. Then 100 μl of 0.1 M DTT were added to each 10 ml of homogenized suspension. Finally, the suspension was centrifuged at 25,000×g for 30 min at 4° C. using F-14 carbon fiber rotor Multifuge 3XR Plus Heraeus centrifuge (Thermo Fisher Scientific), divided to aliquots of 200 μl, frozen by liquid nitrogen and stored at −80° C. for further use.

FIG. 1 is a scheme comparing the lysate preparation according to some embodiments of the invention with that of prior art taught by Pratt, J. M. in Transcription and Translation a practical approach (eds B. D. Hames & S. J. Higgins) Ch. 7, 179-209 (IRL Press 1984).

### Example 2

### Protein Synthesis Using Cell-Free System Based on S30 Lysate Produced According to Some Embodiments of the Invention

Preparation of T7-S30 lysate—as described above.

Reaction Mix According to Some Embodiments of the Invention:

TABLE 2
Ingredients of an exemplary protocol.

| | Final concentration in | |
|---|---|---|
| Ingredient | solution | |
| HEPES-KOH (pH = 8) (Spectrum, New- | 55 | mM |
| Brunswick, NJ, USA) | | |
| Magnesium acetate | 14 | mM |
| Potassium acetate | 50 | mM |
| Ammonium acetate (Merck) | 155 | mM |
| Polyethylene glycol (PEG) | 3% (v/v) | |
| (Sigma-Aldrich) | | |
| 3-phosphoglycerate (3-PGA) (Sigma-Aldrich) | 40 | mM |
| 20 amino acids (Sigma-Aldrich) | 2.5 | mM |
| Adenine triphosphate (ATP) (Sigma-Aldrich) | 1.2 | mM |
| Guanidine triphosphate (GTP) (Sigma- | 1 | mM |
| Aldrich) | | |
| Uridine triphosphate (UTP) (Sigma-Aldrich) | 0.8 | mM |
| S-30 lysate | 30% (v/v) | |
| DNA template | 10 | μg/μL |
| DNase, RNase free H₂O | Complete to 50 | μL |
| Total | 50 | μL |

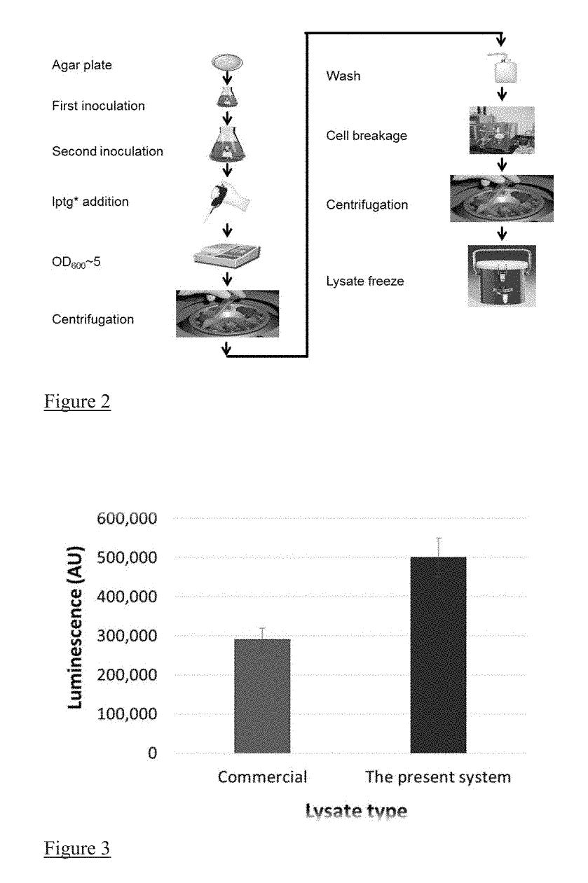

The reaction is conducted in a Thermomixer® (Eppendorf, Gemany) at 30° C. or 37° C. using mild shaking (300 rpm) and lasts for 90 min to 180 min, depends on the produced protein. FIG. 2 is a scheme showing the major elements of the method of protein/RNA production according to some embodiments of the invention.

Renilla Luciferase Production

The protein was produced using the present system with the S30 T7 Control DNA encoding Renilla luciferase, included in S30 T7 high yield protein expression system kit (Promega, Madison, Wis., USA), as the DNA template (SEQ ID NO: 1). The amount of produced protein was measured after 180 min incubation according to a luciferase assay system protocol by Promega (Madison, Wis., USA). In short, Luciferase Cell Culture Lysis Reagent (lysis buffer) was diluted 1:5 with distilled water. The produced protein was diluted 1:40 with the diluted lysis buffer. Luciferase Assay Buffer was added to Luciferase Assay Substrate at a ratio of 1:100. Then 50 μl of lysis buffer and protein mixture were mixed with 50 μl of assay buffer and substrate. Luminescence was determined using a Tecan Infinite200pro plate reader (Tecan, Mannedorf, Switzerland) with a delay time of 10 sec.

Renilla luciferase was produced by the present system with higher efficiency than a commercially available system (Promega) (FIG. 3).

Tyrosinase from Bacillus megaterium (TyrBm) Production

The protein was produced using the present system with a PET9d plasmid encoding TyrBm plasmid as the DNA template. TyrBm of SEQ ID NO: 2 was used. The amount of produced protein was measured after incubation of 180 min. One mM tyrosine and 1 mM Cu⁺² were added to the mixture and incubated for another 30 min at 37° C. The absorbance was determined at 475 nm using a Tecan Infinite200pro plate reader (Tecan, Mannedorf, Switzerland).

Tyrosinase from Bacillus megaterium was produced using the present cell free system according to some embodiments of the invention, as can be observed from FIG. 8. When the DNA template was excluded from the reaction, no protein was produced.

Super Folder GFP (sfGFP)

The protein was produced using the present system with a PET9a plasmid encoding sfGFP as the DNA template (SEQ ID NO: 3). The template was purchased from Sandia BioTech (Albuquerque, N. Mex., USA) and incorporated into pet9a and pet28a. Super folder GFP sequence was inserted to a pet9a and pet28a vector using restriction sites Ndel and BamHI. The protein production was monitored using a Tecan Infinite200pro plate reader (Tecan, Mannedorf, Switzerland) at excitation wavelength of 488 nm and emission of 530 nm.

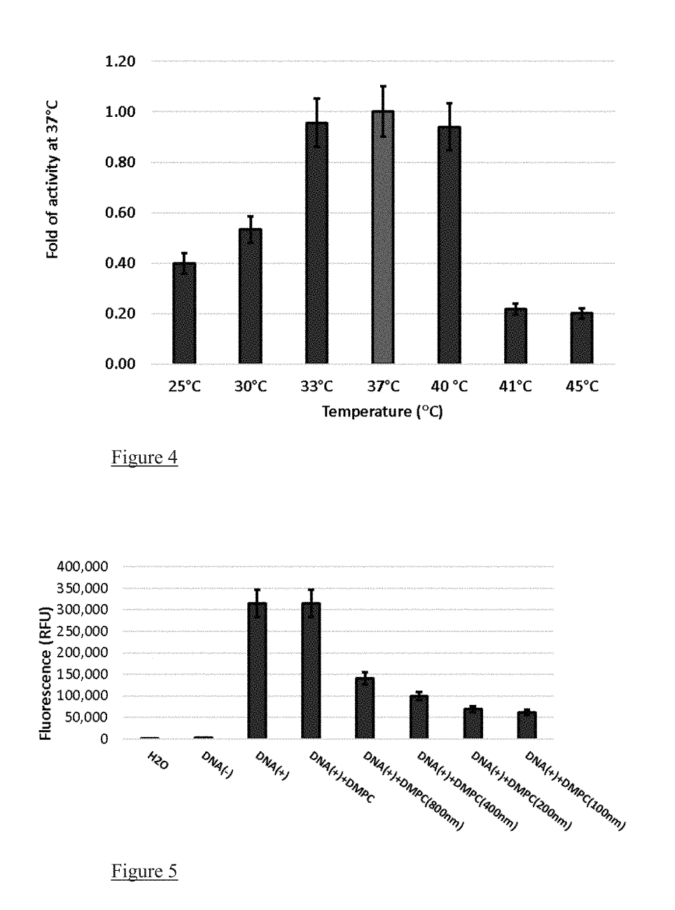

The present system's thermostability was evaluated by performing the reaction for 180 min in different temperatures in the range of 25-45° C. The highest protein producing activity was at 37° C., indicating the present system's optimal thermostability (FIG. 4).

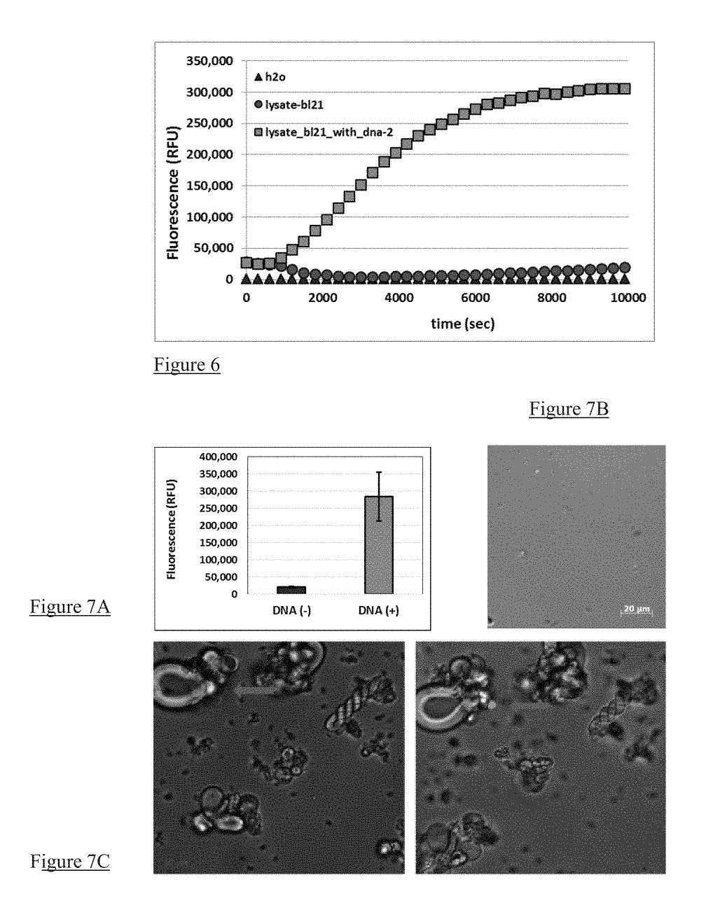

The kinetics of protein production were analyzed by conducting a cell-free reaction in a 96 flat bottom black polystyrol plate (Greiner) at 37° C. The protein production was monitored every 5 min for 166 min using a Tecan Infinite200pro plate reader (Tecan, Mannedorf, Switzerland). As controls, water and a cell-free reaction mixture without a DNA template were monitored in parallel or with the same set points. The appropriate reaction time for production of sfGFP was found to be 120-150 min (FIG. 6).

p50 (NF-kβ Subunit) Production and Evaluation

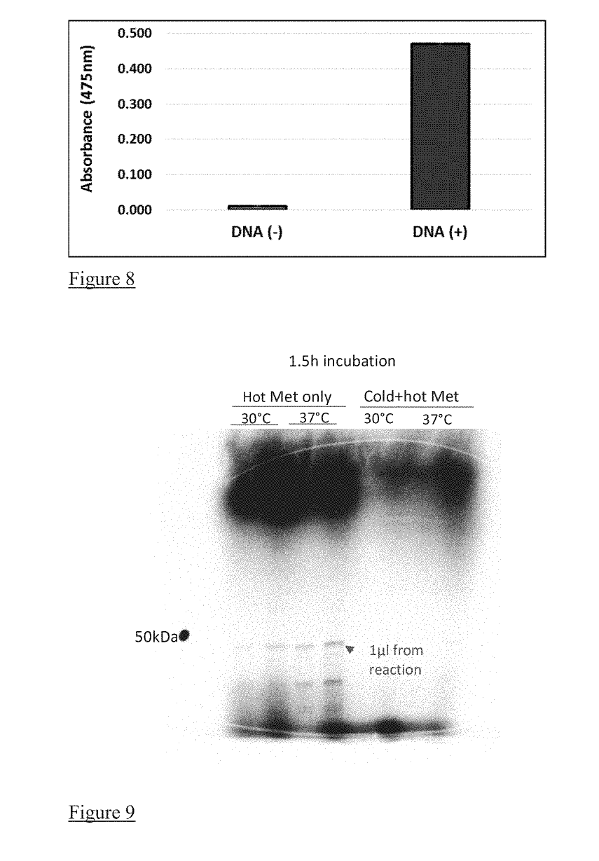

The protein was produced using the present system with template encoding p50 (NF-Kβ subunit, SEQ ID NO: 4). The protein production was performed in 30° C. and 37° C. with [³⁵S] methionine and with or without unlabeled methionine. After 90 min the cell free mixture was mixed with 0.25 volume of ×4 concentrate SDS-PAGE sample buffer and boiled for 10 min at 95° C. Then 1 μL sample was loaded onto a 12% SDS-PAGE gel. Following electrophoresis, the gels were visualized by autoradiography. FIG. 9 presents the production of p50 (NF-Kβ subunit) using the present cell-free system according to some embodiments of the invention. The protein synthesis was more efficient when performed in 37° C. than 30° C.

### Example 3

### Preparation of Particles Containing Cell Free System and Production of Proteins Therein

Particles encapsulating the cell free system were prepared according to Sunami et al., 2006¹¹. Initially, 1,2-dimyristoyl-sn-glycero-3-phosphocholine (DMPC) (Lipoid, Germany) and cholesterol (Sigma-Aldrich) in a molar ratio of 60:40 were dissolved in chloroform. Then the solvent was evaporated using a rotary evaporator (Buchi) enabling the creation of a thin lipid film. The film was hydrated with double distilled water while rotating. The dispersion became milky, indicating the spontaneous formation of lipid vesicles. Nanoscale vesicles were created with stepwise extrusion through polycarbonate membranes (Whatman® Nuclepore™ Track-Etched Membranes) using 400 nm pore size membranes in a 10 mL extrusion system (Northern Lipids, Vancouver, Canada) at 40° C. This step was followed by lyophilization (Labonco). In order to encapsulate the cell free system in the particles, the lyophilized liposomes were rehydrated with a cell free reaction mixture with sfGFP encoding plasmid as the DNA template at a final lipid concentration of 50 mM. The liposome rehydration was carried out using a Thermomixer® (Eppendorf, Germany) at 4° C. using mild shaking (300 rpm) for 20 min to allow the liposomes to form. The non-encapsulated cell free reaction mixture was removed by centrifugation (7,000×g, 1 min, 4° C.) and repeatedly washed with 5% (w/v) dextrose. The final pellet was resuspended in a cell free reaction mixture that excluded the DNA template and the S30 lysate.

Cell Free Protein Production in Particles

The particles were incubated for 2 hr at 37° C. in a Thermomixer® (Eppendorf, Gemany) using mild shaking (300 rpm) to enable protein synthesis within them. The produced sfGFP was observed using a fluorescent microscope (Nikon, Melville, N.Y., USA) under a filter for GFP. The kinetics of protein production inside the particles were monitored using a Zeiss Axiovert 200 inverted fluorescent microscope. The microscope was equipped with an environmental chamber set at 37° C. and the reaction was observed in time intervals of 30 sec.

FIG. 7A shows a positive control for the production of sfGFP using the present cell free system. FIG. 7B presents the production of sfGFP in DMPC-cholesterol particles. FIG. 7A presents an overlay of bright light field with fluorescent field of an online production of sfGFP in DMPC-cholesterol particles. Green spots indicate production of sfGFP within the particles after 10 min.

The effect of an extrusion step on the present cell free system was evaluated by adding 50 mM 1,2-dimyristoyl-sn-glycero-3-phosphocholine (DMPC) (Lipoid) prior to the incubation period 1,2-dimyristoyl-glycero-3-phosphocholine and extruding using a syringe mini extruder (Avanti) and applying polycarbonate membranes with different pore sizes (Whatman® Nuclepore™ Track-Etched Membranes). The amount of produced protein (after incubation period) was evaluated by using a Tecan Infinite200pro plate reader (Tecan, Mannedorf, Switzerland). As can be observed from FIG. 5, there was a decrease in activity at small membrane pore sizes, i.e. smaller particle sizes.

### Example 4

### Therapeutic Protein Synthesis Using Cell-Free System Based on S30 Lysate Produced According to Some Embodiments of the Invention

Pseudomonas Exotoxin A (PE)

The protein was produced using the present system with a pet3 plasmid encoding PE as the gene template (SEQ ID NO: 5). This plasmid was further edited to incorporate a sequence of 6 histidine residues into the protein (PE-his, SEQ ID NO: 6). Purified proteins were used for comparison with the protein produced using the cell free system of some embodiments of the invention.

Purified PE Periplasmatic Production and Purification

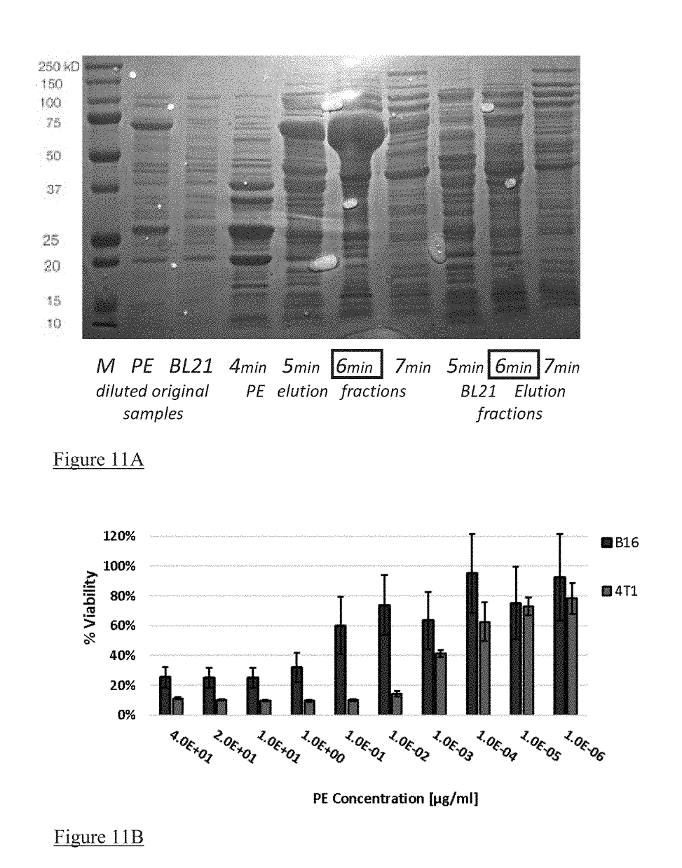

E. coli BL21 transformed with the PE (SEQ ID NO: 6) plasmid glycerol stock (−80° C.) was streaked on Luria Bertani (LB)¹² plate solidified with 1.5% Bacto agar, (Acumedia, Neogen Corporation, MI, USA) supplemented with ampicillin at 100 μg mL⁻¹ (LB-amp100) to maintain the plasmids. A single colony was used to inoculate fresh LB-amp100 media. The culture was grown overnight at 37° C. while shaking at 250 rpm on a TU-400 incubator shaker (Orbital Shaker Incubator, MRC, Holon, Israel) and used as a starter to inoculate fresh Super Broth or Terrific Broth media¹³ (supplemented with 100 μg ml⁻¹ ampicillin) the following day at 1:100 starter:medium ratio. The culture was grown at 37° C. to OD₆₀₀≈2.5, upon which 1 mM of IPTG (Inalco S.P.A., Milano, Italy) was added. The culture was further grown overnight at 30° C. and then centrifuged at 5,000×g for 15 min at 4° C. using MegaFuge centrifuge (Thermo Scientific). The pellet was gently resuspended using sterile glass beads in ice cold 20% sucrose, 30 mM Tris-HCl (pH 7.4), 1 mM EDTA (1:5 buffer to original growth media volume), and left on ice for 15 min. Cells were then centrifuged for 15 min at 6000 rpm (FIBRLITE F15-6x100y rotor, Thermo Scientific) and 4° C. The pellet was gently resuspended in ice cold sterile double-distilled water (1:5 buffer to original growth media volume), and left on ice for 15 min. Following incubation on ice, the periplasmic fraction was collected by centrifuging the cells for 15 min at 7000 rpm and 4° C. (FIBRLITE F15-6x100y rotor, Thermo Scientific). The resulting periplasmic fraction was adjusted to 20 mM Tris-HCl (pH 7.4). The sample was then applied to a Q-sepharose anion exchange column (HiTrap-1 ml, GE Healthcare) and purified using fast protein liquid chromatography (FPLC, AKTA, GE). The buffers used for purification were 20 mM Tris HCl pH 7.4 (Buffer A), used for equilibration and washing, and 1 M NaCl in Buffer A (Buffer B), used as the elution buffer. A gradient of Buffer B from 0-100% over 10 min was used in order to purify the PE. Eluted protein was collected and dialyzed against PBS. E. coli BL21 that was not transformed with a plasmid was produced and purified as described above and the parallel elution fractions were used as a negative control. All the elution fractions were analyzed using an SDS-PAGE 12% gel and Coomassie blue staining, as shown in FIG. 11A. Proteins eluted after 6 min by the anion exchange column were used as the control for further investigations.

PE Cytotoxicitiy:

The cytotoxicity of the protein was determined by an MTT assay (Sigma-Aldrich) as follows: 1×10⁴ cells/well (200 μL) 4T1 cells were seeded in 96-well plates in RPMI complete for 24 h. Then, various concentrations of the purified protein (maximum 10 μg/ml) were added for 24 hours at 37° C. After 24 hours the growth medium was replaced with fresh media. The assay ended with the vacuum draining of the growth media and the addition of 100 μL/well of 1 mg/mL MTT reagent for 1 hour at 37° C. The MTT-formazan crystals were dissolved by the addition of 100 μL/well of the MTT extraction buffer and overnight incubation at 37° C. Cell viability was calculated from the absorbance values read at 570 nm and 690 nm in the Tecan Infinite200pro plate reader (Tecan, Mannedorf, Switzerland). The absorbance values read at 690 nm were used as blanks. The results are expressed as the percentage of living cells with respect to the untreated controls that are processed simultaneously using the following equation:

$$\% Viability = \frac{{Abs}_{570 , treated sample} - {Abs}_{690 , treated sample}}{{Abs}_{570 , untreated sample} - {Abs}_{690 , untreated sample}} \times 100 \% .$$

The therapeutic potency of PE on 4T1 and B16 cells is presented in FIG. 11B.

Cytotoxicity of PE Produced by the Present Cell-Free System:

PE and PE-produced particles were produced by the present cell free system, as previously described. The therapeutic effect of the produced proteins after 120 min incubation at 37° C. was evaluated by an MTT assay, as previously described, by the addition of 5-20% cell free reaction to the growth media. In addition, particles containing purified PE were produced in a similar manner to the cell free particles production method with the addition of purified protein to the lyophilized liposomes instead of the reaction mixture.

The production of PE was determined by western blot as follows. After termination of the 120 min incubation, 30 μL of the cell free reaction mixture were mixed with SDS-PAGE sample buffer (concentrated ×4) and boiled for 10 min at 95° C. Then the samples were loaded onto a 12% SDS-PAGE gel. Following electrophoresis the gels were blotted onto nitrocellulose membranes (Bio-Rad), blocked with 5% nonfat milk powder and probed for 1 hour at Room temprature with anti-PE polyclonal antibodies (Sigma Aldrich, Rehovot, Israel) diluted to 1:5000. After extensive washes the blots were incubated with HRP-conjugated matching secondary antibody (goat anti rabbit) (Genscript) and developed with Clarity™ Western ECL Blotting Substrate (BioRad). The results were visualized using ImageQuant Las4000, GE.

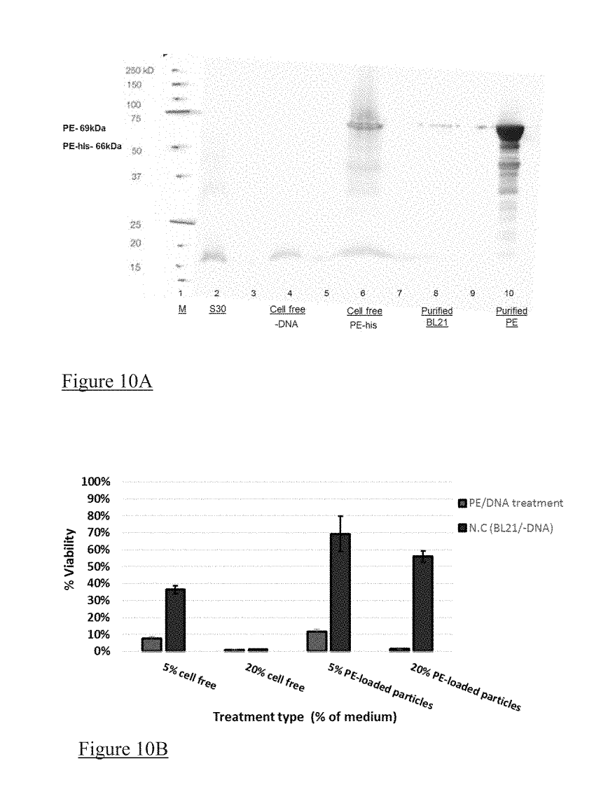

The PE produced by the present cell free system and the PE encapsulated in liposomes present toxicity on 4T1 cell as observed on FIG. 10A. FIG. 10B presents a Western Blot analysis of cell free productions of PE using the present cell free system. Cell free reactions without a DNA template and purified BL21 without PE plasmid were used as negative controls.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for cell-free RNA or protein synthesis, the method comprising synthesizing an RNA or protein of interest in a reaction mixture comprising:
a template DNA encoding the RNA or protein of interest; and
a biological extract of a protease-deficient bacterial cell that has been genetically modified to express a heterologous RNA polymerase, the extract further comprising components necessary for transcription and translation of the protein.

2. A method of producing a reaction mixture for cell-free protein synthesis, the method comprising:
(a) growing a culture of protease-deficient bacterial cells that have been genetically modified to express a heterologous RNA polymerase under conditions which allow expression of the RNA polymerase;
(b) disrupting the protease-deficient bacterial cells to obtain a cell preparation which comprises genomic DNA and broken cells; said disrupting being under conditions which retain functionality of components necessary for transcription and translation of the protein;
(c) removing the genomic DNA; and
(d) freezing the cell preparation following the removing.

3. The method of claim 2 further comprising encapsulating the cell preparation in lipid vesicles.

4. (canceled)

5. The method of claim 2, wherein the disrupting is effected using a pressure homogenizer.

6. The method of claim 2, not comprising dialyzing the cell preparation following the disrupting.

7. The method of claim 2, not comprising freezing and thawing the bacterial cells following step (a) and prior to step (b).

8. The method of claim 2, not comprising adding a protease inhibitor to the cell preparation.

9. The method of claim 2, wherein the removing comprises centrifuging at lower than 30,000×g.

10. A composition comprising an extract of protease-deficient bacterial cells that have been genetically modified to express a heterologous RNA polymerase, the extract comprising components necessary for transcription and translation and being devoid of genomic DNA.

11-17. (canceled)

18. The method of claim 1, wherein the biological extract is present in the reaction mixture at a concentration of 20-40% (v/v).

19. (canceled)

20. The method of claim 1, wherein the reaction mixture further comprises amino acids, rNTPs, H₂O, salt and an ATP-regenerating system.

21. The method of claim 1, wherein the reaction mixture comprises polyethylene glycol.

22. The method of claim 1, wherein the template DNA is a circular DNA.

23-29. (canceled)

30. The method of claim 1, wherein the reaction mixture is as listed in Table 2 above.

31. The method of claim 1, wherein the synthesizing is effected in batch, continuous flow, or semi-continuous flow.

32. The method of claim 1, wherein the protease-deficient bacterial cells are selected from the group consisting of BL21(DE3), BL21(DE3) CodonPlus RIL and variants thereof.

33. The method of claim 1, wherein the protein is a membranal protein.

34. (canceled)

35. The method of claim 3, wherein said lipid vesicles comprise liposomes.
